(19) United States
(12) Patent Application Publication
(10) Pub. No.: US 2006/0241297 A1
(43) Pub. Date: Oct. 26, 2006

(54) Pyridinylamino-pyrimidine derivatives as protein kinase inhibitors

(75) Inventors: Shudong Wang, Nottingham (GB); Christopher Meades, Dundee (GB); Darren Gibson, Dundee (GB); Peter Martin Fischer, Arbroth (GB)

Correspondence Address: LAHIVE & COCKFIELD, 28 STATE STREET, BOSTON, MA, 02109, US

(73) Assignee: Cyclacel Limited, Dundee (GB)

(21) Appl. No.: 11/339,059

(22) Filed: Jan. 25, 2006

Related U.S. Application Data
(63) Continuation of application No. PCT/GB04/03282, filed on Jul. 30, 2004.

(30) Foreign Application Priority Data
Aug. 5, 2003 (GB) 0318347.2
Jul. 30, 2003 (GB) 0317842.3

(51) Int. Cl. C07D 417/14

(52) U.S. Cl. 544/329; 544/331

(57) ABSTRACT

The present invention relates to compounds of formula I, or pharmaceutically acceptable salts thereof, 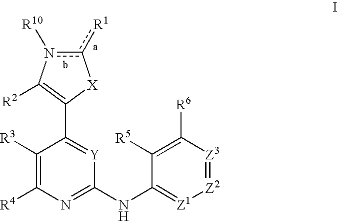 wherein: (A) “a” is a single bond and “b” is a double bond;
R¹ and R² are each independently as defined below;
R¹⁰ is absent; or (B) “a” is a double bond and “b” is a single bond;
R¹ is oxygen;
R² is as defined below; and
R¹⁰ is H or alkyl;
   X is S, O, NH, or NR⁷; Y is N or CR⁸; one of Z¹, Z², and Z³ is N or N⁺R^{a} and the remainder are each independently CR⁷; R¹, R², R⁵ and R⁶ are each independently R⁷; R³ and R⁴ are each independently R⁸; each R⁷ is independently H, halogen, NR^{b}R^{c}, OR^{d} or a hydrocarbyl group optionally substituted by one or more R⁹ groups; each R⁸ is independently H or (CH₂)ₙR⁹, where n is 0 or 1; each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholine, piperidine and piperazine; and R^{a-q} are each independently H or alkyl, wherein said alkyl group is optionally substituted by one or more R⁹ groups; where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine. Further aspects of the invention relate to the use of compounds of formula I in the treatment of proliferative disorders, viral disorders, CNS disorders, strokes, alopecia and/or diabetes.

### RELATED APPLICATIONS

This application is a continuation of PCT/GB2004/003282, filed Jul. 30, 2004, which claims priority to GB 0318347.2, filed on Aug. 5, 2003 and GB0317842.3, filed on Jul. 30, 2003. The entire contents of each of these applications are hereby incorporated herein by reference.

### BACKGROUND TO THE INVENTION

In eukaryotes, all biological functions, including DNA replication, cell cycle progression, energy metabolism, and cell growth and differentiation, are regulated through the reversible phosphorylation of proteins. The phosphorylation state of a protein determines not only its function, subcellular distribution, and stability, but also what other proteins or cellular components it associates with. The balance of specific phosphorylation in the proteome as a whole, as well as of individual members in a biochemical pathway, is thus used by organisms as a strategy to maintain homeostasis in response to an ever-changing environment. The enzymes that carry out these phosphorylation and dephosphorylation steps are protein kinases and phosphatases, respectively.

The eukaryotic protein kinase family is one of the largest in the human genome, comprising some 500 genes [1,2]. The majority of kinases contain a 250-300 amino acid residue catalytic domain with a conserved core structure. This domain comprises a binding pocket for ATP (less frequently GTP), whose terminal phosphate group the kinase transfers covalently to its macromolecular substrates. The phosphate donor is always bound as a complex with a divalent ion (usually Mg²⁺ or Mn²⁺). Another important function of the catalytic domain is the binding and orientation for phosphotransfer of the macromolecular substrate. The catalytic domains present in most kinases are more or less homologous.

A wide variety of molecules capable of inhibiting protein kinase function through antagonising ATP binding are known in the art [3-7]. By way of example, the applicant has previously disclosed 2-anilino-4-heteroaryl-pyrimidine compounds with kinase inhibitory properties, particularly against cyclin-dependent kinases (CDKs) [8-12]. CDKs are serine/threonine protein kinases that associate with various cyclin subunits. These complexes are important for the regulation of eukaryotic cell cycle progression, but also for the regulation of transcription [13,14].

### STATEMENT OF INVENTION

The present invention relates to 4-heteroaryl-2-(pyridinylamino)-pyrimidines and/or 4-heteroaryl-2-(pyridinylamino)-pyridines. In particular, the invention relates to thiazolo-, oxazolo-, and imidazolo-substituted pyrimidine or pyridine compounds and their use in therapy. More specifically, but not exclusively, the invention relates to compounds that have broad therapeutic applications in the treatment of a number of different diseases and/or that are capable of inhibiting one or more protein kinases.

A first aspect of the invention relates to compounds of formula I, or pharmaceutically acceptable salts thereof, 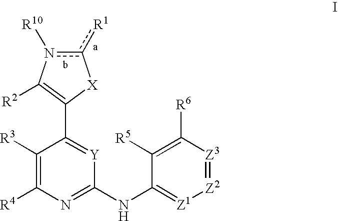
wherein:
(A) “a” is a single bond and “b” is a double bond;
R¹ and R² are each independently as defined below;
R¹⁰ is absent; or
   (B) “a” is a double bond and “b” is a single bond;
R¹ is oxygen;
R² is as defined below; and
R¹⁰ is H or alkyl;
   X is S, O, NH, or NR⁷;
   Y is N or CR⁸;
   one of Z¹, Z², and Z³ is N or N⁺R^{a} and the remainder are each independently CR⁷;
   R¹, R², R⁵ and R⁶ are each independently R⁷;
   R³ and R⁴ are each independently R⁸;
   each R⁷ is independently H, halogen, NR^{b}R^{c}, OR^{d} or a hydrocarbyl group optionally substituted by one or more R⁹ groups;
   each R⁸ is independently H or (CH₂)ₙR⁹, where n is 0 or 1;
   each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
   R^{a-q} are each independently H or alkyl, wherein said alkyl group is optionally substituted by one or more R⁹ groups;
   where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine.

A second aspect of the invention relates to the use of a compound of formula Ia, or a pharmaceutically acceptable salt thereof, 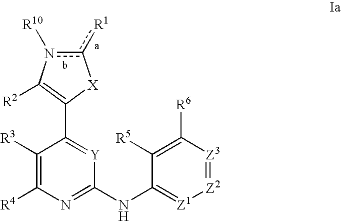
wherein:
(A) “a” is a single bond and “b” is a double bond;
R¹ and R² are each independently as defined below;
R¹⁰ is absent; or
   (B) “a” is a double bond and “b” is a single bond;
R¹ is oxygen;
R² is as defined below; and
R¹⁰ is H or alkyl;
   X is S, O, NH, or NR⁷;
   Y is N or CR⁸;
   one of Z¹, Z², and Z³ is N or N⁺R^{a} and the remainder are each independently CR⁷;
   R¹, R², R⁵ and R⁶ are each independently R⁷;
   R³ and R⁴ are each independently R⁸;
   each R⁷ is independently H, halogen, NR^{b}R^{c}, OR^{d} or a hydrocarbyl group optionally substituted by one or more R⁹ groups;
   each R⁸ is independently H or (CH₂)ₙR⁹, wherein is 0 or 1;
   each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
   R^{a-q} are each independently H or alkyl, wherein said alkyl group is optionally substituted by one or more R⁹ groups;
   in the preparation of a medicament for treating one or more of the following:
a proliferative disorder;
a viral disorder;
a CNS disorder;
a stroke;
alopecia; and
diabetes.

A third aspect of the invention relates to a pharmaceutical composition comprising a compound of formula Ia, or a pharmaceutically acceptable salt thereof, admixed with a pharmaceutically acceptable diluent, excipient or carrier.

A fourth aspect of the invention relates to the use of a compound of formula Ia, or a pharmaceutically acceptable salt thereof, in an assay for identifying further candidate compounds capable of inhibiting one or more of a cyclin dependent kinase, aurora kinase, GSK and a PLK enzyme.

### DETAILED DESCRIPTION

The present invention relates to compounds of formula I and the use of compounds of formula Ia in the preparation of a medicament for treating one or more of a proliferative disorder, a viral disorder, a CNS disorder, a stroke, alopecia and diabetes. Preferred embodiments are the same in respect of compounds of formula I and Ia.

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, alicyclic, heteroalicyclic or alkenyl group. More preferably, the hydrocarbyl group is an aryl, heteroaryl, alkyl, cycloalkyl, aralkyl or alkenyl group. The hydrocarbyl group may be optionally substituted by one or more R⁹ groups.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more R⁹ groups.

As used herein, the term “cycloalkyl” refers to a cyclic alkyl group which may be substituted (mono- or poly-) or unsubstituted. Preferably, the cycloalkyl group is a C₃₋₁₂ cycloalkyl group. Suitable substituents include, for example, one or more R⁹ groups.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group. Suitable substituents include, for example, one or more R⁹ groups as defined above.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R⁹ groups.

As used herein, the term “alicyclic” refers to a cyclic aliphatic group which optionally contains one or more heteroatoms. Preferred alicyclic groups include piperidinyl, piperazinyl, pyrrolidinyl and morpholino.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from O, N and S. Preferred heteroaryl groups include pyrrole, pyrazole, pyrimidine, pyrazine, pyridine, quinoline, thiophene and furan. Again, suitable substituents include, for example, one or more R⁹ groups.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

One preferred embodiment of the invention relates to compounds of formula Ib, or pharmaceutically acceptable salts thereof, 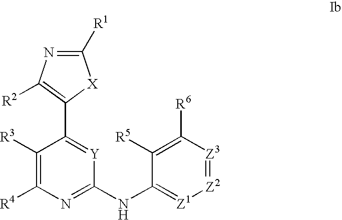
wherein
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups;
where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine.

Another preferred embodiment of the invention relates to compounds of formula Ic, or pharmaceutically acceptable salts thereof, 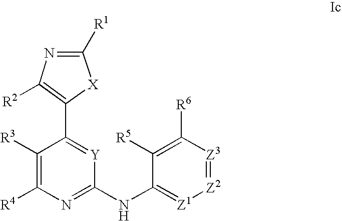
wherein
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholine, piperidine and piperazine; and
R^{a-q} are each independently H or alkyl;
where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine.

In one preferred embodiment, each R⁷ is independently H, halogen, NR^{b}R^{c}, OR^{d} or a saturated or unsaturated group containing between 1 and 20 C atoms, optionally containing one or more heteroatoms selected from from N, S, and O, and optionally substituted with one or more R⁹ groups.

In another preferred embodiment, each R⁷ is independently H, NR^{b}R^{c}, OR^{d} or a saturated or unsaturated group containing between 1 and 20 carbon atoms, optionally containing one or more heteroatoms selected from from N, S, and O, and optionally substituted with one or more R⁹ groups.

More preferably, each R⁷ is independently H, halogen, NR^{b}R^{c}, OR^{d} or is an alkyl, cycloalkyl, aryl or aralkyl group, each of which optionally contain one to six heteroatoms selected from N, S and O, and each of which is optionally substituted by one to six R⁹ groups.

Even more preferably, each R⁷ is independently H, NR^{b}R^{c}, OR^{d} or is an alkyl, cycloalkyl, aryl, alicyclic or aralkyl group, optionally containing one to six heteroatoms selected from N, S and O, and optionally substituted by one to six R⁹ groups.

Even more preferably, each R⁷ is independently H, NR^{b}R^{c}, OR^{d} or is an alkyl, cycloalkyl, aryl or aralkyl group, optionally containing one to six heteroatoms selected from N, S and O, and optionally substituted by one to six R⁹ groups.

In one preferred embodiment, each R⁷ is independently selected from H, OR^{d}, NR^{b}R^{c}, halogen and an alicyclic group optionally comprising one or more heteroatoms and which is optionally substituted by one or more R⁹ groups.

In another preferred embodiment, each R⁷ is independently selected from H, OR^{d}, NR^{b}R^{c}, halogen and an alicyclic group selected from pyrrolidinyl, piperidinyl, morpholino and piperazinyl, each of which is optionally substituted by one or more R⁹ groups.

In another preferred embodiment, each R⁷ is independently selected from Me, Cl, OMe, OEt, NH₂, NHMe, NHEt, NMe₂, N-pyrrolidinyl, N-piperidinyl, N-morpholino and N-piperazinyl.

More preferably still, each R⁷ is independently selected from Me, OMe, OEt, NH₂, NHMe, NHEt and NMe₂.

In another preferred embodiment, R^{a-q} are each independently H, Me or Et, said Me or Et groups being optionally substituted by one or more R⁹ groups.

In one preferred embodiment, R^{a-q} are each independently H, Me or Et.

Preferably, R⁹ is selected from H, halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

More preferably, R⁹ is selected from OMe, halogen, NH₂, CN, NO₂, CF₃, OEt, NMe₂, NHMe and OH.

In one preferred embodiment:

one of Z² and Z³ is N or N⁺R^{a}; and

Z¹ and the other of Z² and Z³ are each independently CR⁷.

In one particularly preferred embodiment, Z² is N or NR^{a+} and Z¹ and Z³ are each independently CR⁷.

More preferably, Z² is N or NR^{a+}, Z¹ is C—H and Z³ is C—Cl or C—OMe.

In one preferred embodiment of the invention, Y is N, i.e. the compound of formula I or Ia is a 4-heteroaryl-2-pyridinyl-pyrimidine derivative.

In another preferred embodiment of the invention, Y is CR⁸, i.e. the compound of formula I or Ia is a 4-heteroaryl-2-pyridinyl-pyridine derivative.

In one preferred embodiment of the invention, X is S, O or NH.

In one especially preferred embodiment of the invention, X is S, i.e. the compound of formula I or Ia is a 4-thiazolyl-substituted-2-pyridinyl-pyridine derivative or a 4-thiazolyl-substituted-2-pyridinyl-pyrimidine derivative.

In one preferred embodiment of the invention, R¹ is selected from Me, OMe, OEt, NH₂, NHMe, NHEt and NMe₂.

In another preferred embodiment of the invention, R² is Me.

In yet another preferred embodiment, R³, R⁴, R⁵ and R⁶ are all H.

Another preferred embodiment of the invention relates to compounds of formula Id, or pharmaceutically acceptable salts thereof, 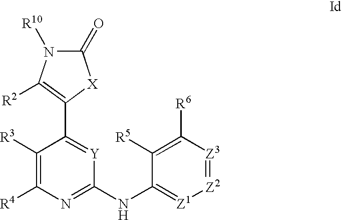
wherein R²⁻⁶, R¹⁰, X, Y, Z¹, Z² and Z³ are as defined above.

In one especially preferred embodiment of the invention, the compound is selected from the following:
[4-(2,4-Dimethyl-thiazol-5-yl)-pyrimidin-2-yl]-pyridin-3-yl-amine [1];
3-[4-(2,4-Dimethyl-thiazol-5-yl)-pyrimidin-2-ylamino]-1-methyl-pyridinium [2]; (6-Chloro-pyridin-3-yl)-[4-(2,4-dimethyl-thiazol-S-yl)-pyrimidin-2-yl]-amine [3];
5-[2-(6-chloro-pyridin-3-ylamino)-pyrimidin-4-yl]-3,4-dimethyl-3H-thiazol-2-one [4];
[4-(2-Amino-4-methyl-thiazol-5-yl)-pyrimidin-2-yl]-(6-methoxy-pyridin-3-yl)-amine [5];
[4-(2,4-Dimethyl-thiazol-5-yl)-pyrimidin-2-yl]-(6-methoxy-pyridin-3-yl)-amine [6];
[4-(2-Amino-4-methyl-thiazol-5-yl)-pyrimidin-2-yl]-(6-chloro-pyridin-3-yl)-amine [7];
(6-Methoxy-pyridin-3-yl)-[4-(4-methyl-2-methylamino-thiazol-5-yl)-pyrimidin-2-yl]-amine [8];
(6-Chloro-pyridin-3-yl)-[4-(4-methyl-2-methylamino-thiazol-5-yl)-pyrimidin-2-yl]-amine [9];
[4-(2-Dimethylamino-4-methyl-thiazol-5-yl)-pyrimidin-2-yl]-(6-methoxy-pyridin-3-yl)-amine [10];
3-Ethyl-S-[2-(6-methoxy-pyridin-3-ylamino)-pyrimidin-4-yl]-4-methyl-3H-thiazol-2-one [11];
[4-(2-Ethylamino-4-methyl-thiazol-S-yl)-pyrimidin-2-yl]-(6-methoxy-pyridin-3-yl)-amine [12];
{4-[2-(2-Methoxy-ethylamino)-4-methyl-thiazol-5-yl]-pyrimidin-2-yl}-(6-methoxy-pyridin-3-yl)-amine [13];
[4-(2,4-Dimethyl-thiazol-5-yl)-pyrimidin-2-yl]-(6-pyrrolidin-1-yl-pyridin-3-yl)-amine [14];
[4-(4-Methyl-2-methylamino-thiazol-5-yl)-pyrimidin-2-yl]-[6-(4-methyl-piperazin-1-yl)-pyridin-3-yl]-amine [15]; and
(6-Methoxy-pyridin-3-yl)-[4-(4-methyl-2-morpholin-4-yl-thiazol-5-yl)-pyrimidin-2-yl]-amine [16].

In another particularly preferred embodiment, the compound of the invention is selected from the following:
[4-(2,4-Dimethyl-thiazol-5-yl)-pyrimidin-2-yl]-pyridin-3-yl-amine [1];
3-[4-(2,4-Dimethyl-thiazol-5-yl)-pyrimidin-2-ylamino]-1-methyl-pyridinium [2];
(6-Chloro-pyridin-3-yl)-[4-(2,4-dimethyl-thiazol-5-yl)-pyrimidin-2-yl]-amine [3];
(6-Chloro-pyridin-3-yl)-[4-(2-methoxy-4-methyl-thiazol-5-yl)-pyrimidin-2-yl]-amine [4];
[4-(2-Amino-4-methyl-thiazol-5-yl)-pyrimidin-2-yl]-(6-methoxy-pyridin-3-yl)-amine [5];
[4-(2,4-Dimethyl-thiazol-5-yl)-pyrimidin-2-yl]-(6-methoxy-pyridin-3-yl)-amine [6];
[4-(2-Amino-4-methyl-thiazol-5-yl)-pyrimidin-2-yl]-(6-chloro-pyridin-3-yl)-amine [7];
(6-Methoxy-pyridin-3-yl)-[4-(4-methyl-2-methylamino-thiazol-5-yl)-pyrimidin-2-yl]-amine [8];
(6-Chloro-pyridin-3-yl)-[4-(4-methyl-2-methylamino-thiazol-5-yl)-pyrimidin-2-yl]-amine [9];
[4-(2-Dimethylamino-4-methyl-thiazol-5-yl)-pyrimidin-2-yl]-(6-methoxy-pyridin-3-yl)-amine [10];
[4-(2-Ethoxy-4-methyl-thiazol-5-yl)-pyrimidin-2-yl]-(6-methoxy-pyridin-3-yl)-amine [11]; and
[4-(2-Ethylamino-4-methyl-thiazol-5-yl)-pyrimidin-2-yl]-(6-methoxy-pyridin-3-yl)-amine [12].

In one preferred embodiment, the compound is capable of inhibiting one or more protein kinases selected from CDK1/cyclin B, CDK2/cyclin A, CDK2/cyclin E, CDK4/cyclin Dl, CDK7/cyclin H, CDK9/cyclin Ti, aurora kinase, GSK3β and PLK1, as measured by the appropriate assay. Details of the various kinase assays may be found in the accompanying examples section and will be familiar to those skilled in the art.

More preferably, the compound exhibits an IC₅₀ value (for kinase inhibition) of less than 1 μM. Thus, in one preferred embodiment, the compound of formula I or Ia is selected from the following: [1], [3], [4], [5], [6], [7], [8], [9], [10], [11], [12], [13], [14] and [15].

More preferably still, the compound exhibits an IC₅₀ value (for kinase inhibition) of less than 0.1 μM. Thus, in one preferred embodiment, the compound of formula I or Ia is selected from the following: [1], [4], [5], [7], [8], [9], [10], [11], [12] and [15].

In one preferred embodiment, the compound is selected from the following: [4], [5], [7], [8] and [11].

Even more preferably, the compound exhibits an IC₅₀ value (for kinase inhibition) of less than 0.01 μM. Thus, in one preferred embodiment, the compound of formula I or Ia is selected from the following: [5], [7] and [8].

In one preferred embodiment, the invention relates to compounds that are capable of exhibiting an antiproliferative effect against one or more transformed human cell lines in vitro as measured by a 72-h MTT cytotoxicity assay. Preferably, the compound is selected from the following: [1]-[10], [12] and [14] as defined above.

Peferably, the compound of the invention is capable of exhibiting an IC₅₀ value (average) of less than 10 μM against one or more transformed human cell lines in vitro as measured by a 72-h MTT cytotoxicity assay. Thus, preferably, the compound of formula I or Ia is selected from the following: [1], [3], [4], [5], [6], [7], [8], [9], [10], [12] and [14].

Even more preferably, the compound of the invention is capable of exhibiting an IC₅₀ value (average) of less than 5 μM against one or more transformed human cell lines in vitro as measured by a 72-h MTT cytotoxicity assay. Thus, preferably, the compound of formula I or Ia is selected from the following: [1], [4], [5], [6], [7], [8], [9], [10], [12] and [14].

More preferably still, the compound is capable of exhibiting an IC₅₀ value (average) of less than 2.5 μM, and even more preferably less than 2 μM against one or more transformed human cell lines in vitro as measured by a 72-h MTT cytotoxicity assay. Thus, preferably, the compound of formula I or Ia is selected from the following: [4], [8], [10], [12] and [14].

More preferably still, the compound is capable of exhibiting an IC₅₀ value (average) of less than 1 μM against one or more transformed human cell lines in vitro as measured by a 72-h MTT cytotoxicity assay. Thus, preferably, the compound of formula I or Ia is compound [14].

Therapeutic Use

The compounds of formula Ia have been found to possess anti-proliferative activity and are therefore believed to be of use in the treatment of proliferative disorders such as cancers, leukaemias and other disorders associated with uncontrolled cellular proliferation such as psoriasis and restenosis. As defined herein, an anti-proliferative effect within the scope of the present invention may be demonstrated by the ability to inhibit cell proliferation in an in vitro whole cell assay, for example using any of the cell lines A549, HT29 or Saos-2 Using such assays it may be determined whether a compound is anti-proliferative in the context of the present invention.

On preferred embodiment of the present invention therefore relates to the use of one or more compounds of formula Ia in the preparation of a medicament for treating a proliferative disorder.

As used herein the phrase “preparation of a medicament” includes the use of a compound of formula Ia directly as the medicament in addition to its use in a screening programme for further therapeutic agents or in any stage of the manufacture of such a medicament.

Preferably, the proliferative disorder is a cancer or leukaemia. The term proliferative disorder is used herein in a broad sense to include any disorder that requires control of the cell cycle, for example cardiovascular disorders such as restenosis, cardiomyopathy and myocardial infarction, auto-immune disorders such as glomerulonephritis and rheumatoid arthritis, dermatological disorders such as psoriasis, anti-inflammatory, anti-fungal, antiparasitic disorders such as malaria, emphysema, alopecia, and chronic obstructive pulmonary disorder. In these disorders, the compounds of the present invention may induce apoptosis or maintain stasis within the desired cells as required.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment of the invention, the compound of formula Ia is administered in an amount sufficient to inhibit at least one CDK enzyme.

Preferably, the compound of formula Ia is administered in an amount sufficient to inhibit at least one of CDK2 and/or CDK4.

Another aspect of the invention relates to the use of a compound of formula Ia in the preparation of a medicament for treating a viral disorder, such as human cytomegalovirus (HCMV), herpes simplex virus type I (HSV-1), human immunodeficiency virus type I (HIV-1), and varicella zoster virus (VZV).

In a more preferred embodiment of the invention, the compound of formula Ia is administered in an amount sufficient to inhibit one or more of the host cell CDKs involved in viral replication, i.e. CDK2, CDK7, CDK8, and CDK9 [23].

As defined herein, an anti-viral effect within the scope of the present invention may be demonstrated by the ability to inhibit CDK2, CDK7, CDK8 or CDK9.

In a particularly preferred embodiment, the invention relates to the use of one or more compounds of formula Ia in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

Another aspect of the invention relates to a method of treating a CDK-dependent disorder, said method comprising administering to a subject in need thereof, a compound of formula Ia, or a pharmaceutically acceptable salt thereof, as defined above in an amount sufficient to inhibit a cyclin dependent kinase.

Preferably, the CDK-dependent disorder is a viral disorder, or a proliferative disorder, more preferably cancer.

Another aspect of the invention relates to the use of compounds of formula Ia, or pharmaceutically acceptable salts thereof, in the preparation of a medicament for treating diabetes.

In a particularly preferred embodiment, the diabetes is type II diabetes.

GSK3 is one of several protein kinases that phosphorylate glycogen synthase (GS). The stimulation of glycogen synthesis by insulin in skeletal muscle results from the dephosphorylation and activation of GS. GSK3's action on GS thus results in the latter's deactivation and thus suppression of the conversion of glucose into glycogen in muscles.

Type II diabetes (non-insulin dependent diabetes mellitus) is a multi-factorial disease. Hyperglycaemia is due to insulin resistance in the liver, muscles, and other tissues, coupled with impaired secretion of insulin. Skeletal muscle is the main site for insulin-stimulated glucose uptake, there it is either removed from circulation or converted to glycogen. Muscle glycogen deposition is the main determinant in glucose homeostasis and type II diabetics have defective muscle glycogen storage. There is evidence that an increase in GSK3 activity is important in type II diabetes [24]. Furthermore, it has been demonstrated that GSK3 is over-expressed in muscle cells of type II diabetics and that an inverse correlation exists between skeletal muscle GSK3 activity and insulin action [25].

GSK3 inhibition is therefore of therapeutic significance in the treatment of diabetes, particularly type II, and diabetic neuropathy.

It is notable that GSK3 is known to phosphorylate many substrates other than GS, and is thus involved in the regulation of multiple biochemical pathways. For example, GSK is highly expressed in the central and peripheral nervous systems.

Another aspect of the invention therefore relates to the use of compounds of formula Ia, or pharmaceutically acceptable salts thereof, in the preparation of a medicament for treating a CNS disorders, for example neurodegenerative disorders.

Preferably, the CNS disorder is Alzheimer's disease.

Tau is a GSK-3 substrate which has been implicated in the etiology of Alzheimer's disease. In healthy nerve cells, Tau co-assembles with tubulin into microtubules. However, in Alzheimer's disease, tau forms large tangles of filaments, which disrupt the microtubule structures in the nerve cell, thereby impairing the transport of nutrients as well as the transmission of neuronal messages.

Without wishing to be bound by theory, it is believed that GSK3 inhibitors may be able to prevent and/or reverse the abnormal hyperphosphorylation of the microtubule-associated protein tau that is an invariant feature of Alzheimer's disease and a number of other neurodegenerative diseases, such as progressive supranuclear palsy, corticobasal degeneration and Pick's disease. Mutations in the tau gene cause inherited forms of fronto-temporal dementia, further underscoring the relevance of tau protein dysfunction for the neurodegenerative process [26].

Another aspect of the invention relates to the use of compounds of formula Ia, or pharmaceutically acceptable salts thereof, in the preparation of a medicament for treating bipolar disorder.

Yet another aspect of the invention relates to the use of compounds of formula Ia, or pharmaceutically acceptable salts thereof, in the preparation of a medicament for treating a stroke.

Reducing neuronal apoptosis is an important therapeutic goal in the context of head trauma, stroke, epilepsy, and motor neuron disease [27]. Therefore, GSK3 as a pro-apoptotic factor in neuronal cells makes this protein kinase an attractive therapeutic target for the design of inhibitory drugs to treat these diseases.

Yet another aspect of the invention relates to the use of compounds of formula Ia, or pharmaceutically acceptable salts thereof, in the preparation of a medicament for treating alopecia.

Hair growth is controlled by the Wnt signalling pathway, in particular Wnt-3. In tissue-culture model systems of the skin, the expression of non-degradable mutants of β-catenin leads to a dramatic increase in the population of putative stem cells, which have greater proliferative potential [28]. This population of stem cells expresses a higher level of non-cadherin-associated β-catenin [29], which may contribute to their high proliferative potential. Moreover, transgenic mice overexpressing a truncated β-catenin in the skin undergo de novo hair-follicle morphogenesis, which normally is only established during embryogenesis. The ectopic application of GSK3 inhibitors may therefore be therapeutically useful in the treatment of baldness and in restoring hair growth following chemotherapy-induced alopecia.

In a preferred embodiment of the invention, the compound of formula Ia, or pharmaceutically acceptable salt thereof, is administered in an amount sufficient to inhibit GSK30.

More preferably, the compound of formula Ia, or pharmaceutically acceptable salt thereof, is administered in an amount sufficient to inhibit GSK3β.

Another aspect of the invention relates to a method of treating a GSK3-dependent disorder, said method comprising administering to a subject in need thereof, a compound of formula Ia, or a pharmaceutically acceptable salt thereof, as defined above in an amount sufficient to inhibit GSK3. Preferably, the compound of formula Ia, or pharmaceutically acceptable salt thereof, is administered in an amount sufficient to inhibit GSK30.

Preferably, the GSK3-dependent disorder is diabetes.

In one embodiment of the invention, the compound of formula Ia is administered in an amount sufficient to inhibit at least one PLK enzyme.

The polo-like kinases (PLKs) constitute a family of serine/threonine protein kinases. Mitotic Drosophila melanogaster mutants at the polo locus display spindle abnormalities [30] and polo was found to encode a mitotic kinase [31]. In humans, there exist three closely related PLKs [32]. They contain a highly homologous amino-terminal catalytic kinase domain and their carboxyl termini contain two or three conserved regions, the polo boxes. The function of the polo boxes remains incompletely understood but they are implicated in the targeting of PLKs to subcellular compartments [33,34], mediation of interactions with other proteins [35], or may constitute part of an autoregulatory domain [36]. Furthermore, the polo box-dependent PLK1 activity is required for proper metaphase/anaphase transition and cytokinesis [37,38].

Studies have shown that human PLKs regulate some fundamental aspects of mitosis [39,40]. In particular, PLK1 activity is believed to be necessary for the functional maturation of centrosomes in late G2/early prophase and subsequent establishment of a bipolar spindle. Depletion of cellular PLK1 through the small interfering RNA (siRNA) technique has also confirmed that this protein is required for multiple mitotic processes and completion of cytokinesis [41].

In a more preferred embodiment of the invention, the compound of formula Ia is administered in an amount sufficient to inhibit PLK1.

Of the three human PLKs, PLK1 is the best characterized; it regulates a number of cell division cycle effects, including the onset of mitosis [42,43], DNA-damage checkpoint activation [44,45], regulation of the anaphase promoting complex [46-48], phosphorylation of the proteasome [49], and centrosome duplication and maturation [50].

Specifically, initiation of mitosis requires activation of M-phase promoting factor (MPF), the complex between the cyclin dependent kinase CDK1 and B-type cyclins [51]. The latter accumulate during the S and G2 phases of the cell cycle and promote the inhibitory phosphorylation of the MPF complex by WEE1, MIK1, and MYT1 kinases. At the end of the G2 phase, corresponding dephosphorylation by the dual-specificity phosphatase CDC25C triggers the activation of MPF [52]. In interphase, cyclin B localizes to the cytoplasm [53], it then becomes phosphorylated during prophase and this event causes nuclear translocation [54,55]. The nuclear accumulation of active MPF during prophase is thought to be important for initiating M-phase events [56]. However, nuclear MPF is kept inactive by WEE1 unless counteracted by CDC25C. The phosphatase CDC25C itself, localized to the cytoplasm during interphase, accumulates in the nucleus in prophase [57-59]. The nuclear entry of both cyclin B [60] and CDC25C [61] are promoted through phosphorylation by PLK1 [43]. This kinase is an important regulator of M-phase initiation.

In one particularly preferred embodiment, the compounds of formula Ia are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of formula Ia is administered in an amount sufficient to inhibit PLK2 and/or PLK3.

Mammalian PLK2 (also known as SNK) and PLK3 (also known as PRK and FNK) were originally shown to be immediate early gene products. PLK3 kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [62]. PLK2 is the least well understood homologue of the three PLKs. Both PLK2 and PLK3 may have additional important post-mitotic functions [35].

A further aspect of the invention relates to a method of treating a PLK-dependent disorder, said method comprising administering to a subject in need thereof, a compound of formula Ia, or a pharmaceutically acceptable salt thereof, as defined above in an amount sufficient to inhibit PLK.

Preferably, the PLK-dependent disorder is a proliferative disorder, more preferably, cancer.

In another preferred embodiment of the invention, the compound of formula Ia is administered in an amount sufficient to inhibit an aurora kinase.

Another aspect of the invention relates to a method of treating an aurora kinase-dependent disorder, said method comprising administering to a subject in need thereof, a compound of formula Ia, or a pharmaceutically acceptable salt thereof, as defined above in an amount sufficient to inhibit aurora kinase.

Preferably, the aurora kinase dependent disorder is a viral disorder as defined above.

Pharmaceutical Compositions

Another aspect of the invention relates to a pharmaceutical composition comprising a compound of formula I and Ia as defined above admixed with one or more pharmaceutically acceptable diluents, excipients or carriers. Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and PJ Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of formula I, Ia, Ib, Ic or Id can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of formula I, Ia, Ib, Ic or Id. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agent or pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes the use of solvate forms of the compounds of the present invention. The terms used in the claims encompass these forms.

Polymorphs

The invention furthermore relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of the present invention in prodrug form. Such prodrugs are generally compounds of formula I, Ia, Ib, Ic or Id wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient.

Combinations

In a particularly preferred embodiment, the one or more compounds of the invention are administered in combination with one or more other therapeutically active agents, for example, existing drugs available on the market. In such cases, the compounds of the invention may be administered consecutively, simultaneously or sequentially with the one or more other active agents.

By way of example, it is known that anticancer drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in early tumor cells which would have been otherwise responsive to initial chemotherapy with a single agent. An example of the use of biochemical interactions in selecting drug combinations is demonstrated by the administration of leucovorin to increase the binding of an active intracellular metabolite of 5-fluorouracil to its target, thymidylate synthase, thus increasing its cytotoxic effects.

Numerous combinations are used in current treatments of cancer and leukemia. A more extensive review of medical practices may be found in “Oncologic Therapies” edited by E. E. Vokes and H. M. Golomb, published by Springer.

Beneficial combinations may be suggested by studying the growth inhibitory activity of the test compounds with agents known or suspected of being valuable in the treatment of a particular cancer initially or cell lines derived from that cancer. This procedure can also be used to determine the order of administration of the agents, i.e. before, simultaneously, or after delivery. Such scheduling may be a feature of all the cycle acting agents identified herein.

Assays

Another aspect of the invention relates to the use of a compound of the invention in an assay for identifying further candidate compounds capable of inhibiting one or more protein kinases.

Another aspect of the invention relates to the use of a compound of the invention in an assay for identifying further candidate compounds capable of inhibiting one or more of a cyclin dependent kinase, aurora kinase, GSK and PLK.

Preferably, the assay is a competitive binding assay.

More preferably, the competitive binding assay comprises contacting a compound of the invention with a protein kinase and a candidate compound and detecting any change in the interaction between the compound of the invention and the protein kinase.

One aspect of the invention relates to a process comprising the steps of:

(a) performing an assay method described hereinabove;

(b) identifying one or more ligands capable of binding to a ligand binding domain; and

(c) preparing a quantity of said one or more ligands.

Another aspect of the invention provides a process comprising the steps of:

(a) performing an assay method described hereinabove;

(b) identifying one or more ligands capable of binding to a ligand binding domain; and

(c) preparing a pharmaceutical composition comprising said one or more ligands.

Another aspect of the invention provides a process comprising the steps of:

(a) performing an assay method described hereinabove;

(b) identifying one or more ligands capable of binding to a ligand binding domain;

(c) modifying said one or more ligands capable of binding to a ligand binding domain;

(d) performing the assay method described hereinabove;

(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of proliferative disorders, viral disorders, a CNS disorder, stroke, alopecia and diabetes.

Preferably, said candidate compound is generated by conventional SAR modification of a compound of the invention.

As used herein, the term “conventional SAR modification” refers to standard methods known in the art for varying a given compound by way of chemical derivatisation.

The above methods may be used to screen for a ligand useful as an inhibitor of one or more protein kinases.

Synthesis

Compounds of general structure I and Ia can be prepared by any method known in the art. Two convenient synthetic routes of preparing compounds of formula I and Ia are shown in Scheme I below:

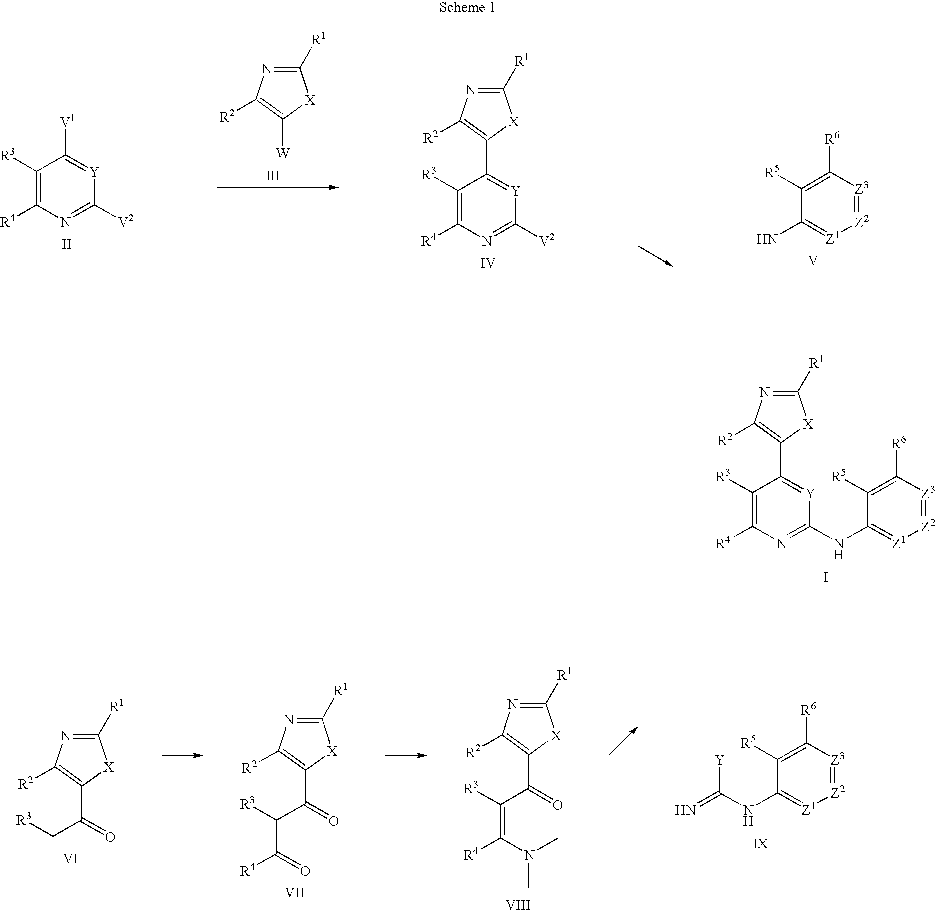

Palladium-catalysed cross-coupling of heteroaryl boronic acids (III, W═B(OH)₂) or their derivatives [63] with 2,4-dihalogenated pyrimidines (II; e.g. V¹═V²═Cl, Y═N) or pyridines (II; e.g. V¹═I, V²═Cl, Br, or F, Y═CR⁸) [64] affords 4-heteroarylated 2-halogenopyrimidines IV, which are aminated with anilines V. Alternatively, in the case where Y is N, acylheterocyclic compounds VI, which can be prepared from heterocyclic precursor compounds e.g. through Friedel-Crafts acylation, are further acylated, e.g. with R⁴COCl, to provide the diketones VII. These in turn are enaminated to VIII [65], followed by condensation with arylguanidines IX [66].

Thus, another aspect of the invention relates to a process for preparing a compound of formula Ib as defined above, said process comprising the steps of: 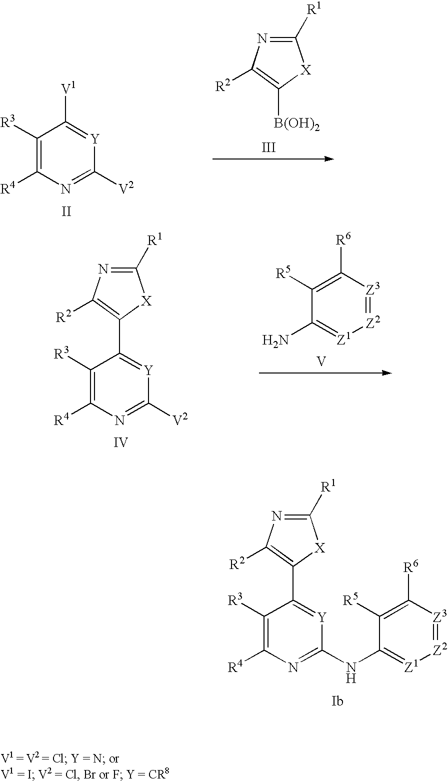
(i) reacting a heteroaryl boronic acid of formula III with a 2,4-dihalogenated pyrimidine or pyridine of formula II to form a compound of formula IV;
(ii) reacting said compound of formula IV with an aniline of formula V to form a compound of formula Ib.

Yet another aspect of the invention relates to a process for preparing a compound of formula Ib as defined above, said process comprising the steps of: 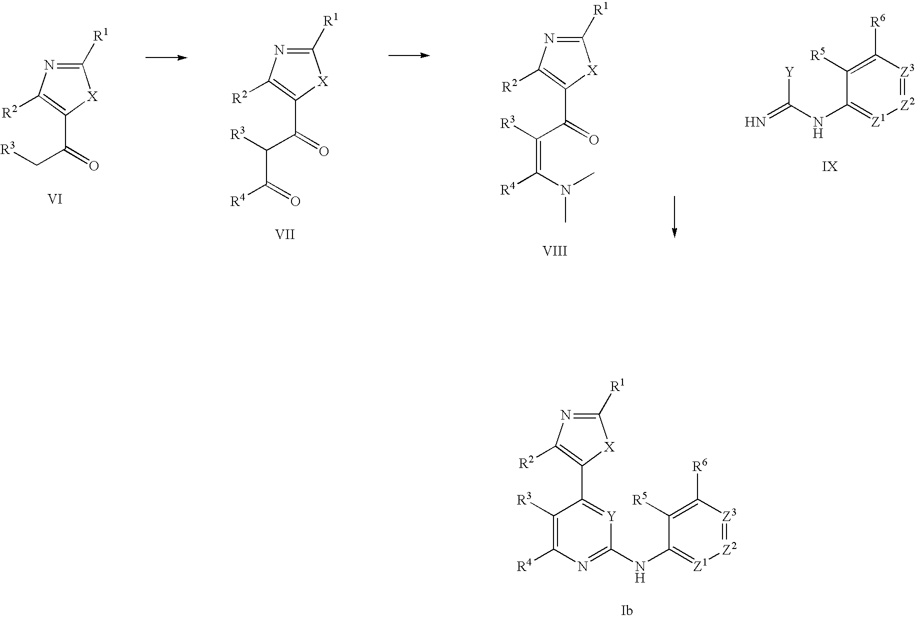
(I) reacting an acylheterocyclic compound of formula VI with R⁴COCl to form a diketone of formula VII;
(ii) converting said diketone of formula VII to a compound of formula VIII;
(iii) reacting said compound of formula VIII with an arylguanidine of formula IX to form said compound of formula Ib.

The present invention is further described by way of the following non-limiting examples.

### EXAMPLES

General

HPLC retention times (t_{R}) were measured using Vydac 218TP54 columns (C₁₈ reversed-phase stationary phase; 4.5×250 mm columns), eluted at 1 mL/min with a linear gradient of acetonitrile in water (containing 0.1% CF₃COOH) as indicated, followed by isocratic elution. UV monitors (254 nm) were used. All purification work, unless otherwise stated, was performed using silica gel 60A (particle size 35-70 micron). ¹H-NMR spectra were recorded using a 500 MHz instrument. Chemical shifts are given in ppm using TMS as standard and coupling constants (j) are stated in Hz. Mass spectra were recorded under positive or negative ion electrospray (ESI) or delayed extraction matrix-assisted laser desorption ionisation time-of-flight (DE MALDI-TOF) conditions.

The structures of selected compounds of the invention are shown in Table 1.

### [4-(2,4-Dimethyl-thiazol-5-yl)-pyrimidin-2-yl]-pyridin-3-yl-amine (1)

A mixture of 3-dimethylamino-1-(2,4-dimethylthiazol-5-yl)-propenone (0.30 g, 1.56 mmol), N-pyridin-3-yl-guanidine dihydrochloride (0.39 g, 1.87 mmol), and K₂CO₃ (0.54 g, 3.93 mmol) in 2-methoxyethanol (9 mL) was heated at reflux for 18 h. The mixture was evaporated to dryness and the residue purified by SiO₂ gel chromatography. After recrystallisation from EtOAc the title compound (0.11 g, 24%) was obtained as a pale brown solid: m.p. 159-162° C. ¹H-NMR (DMSO-d₆): δ 2.63 (s, 3H, CH₃), 2.65 (s, 3H, CH₃), 7.14 (d, 1H, J=5.4 Hz, pyrimidine-H), 7.32 (dd, 1H, J=8.1, 4.4 Hz, pyridine-H), 8.16-8.19 (m, 2H, pyridine-H), 8.55 (d, 1H, J=5.4 Hz, pyrimidine-H), 8.92 (d, 1H, J=2.4 Hz, pyridine-H), 9.86 (bs, 1H, NH). MS (DE MALDI-TOF) m/z 283.17 [M], C₁₄H₁₃N₅S requires 283.35.

### 3-[4-(2,4-Dimethyl-thiazol-5-yl)-pyrimidin-2-ylamino]-1-methyl-pyridinium (2)

A solution of compound 1 (0.035 g, 0.12 mmol) in anh. Me₂CO (6 mL) was treated with iodomethane (12 μL, 0.19 mmol). After heating at reflux for 18 h, the reaction mixture was cooled to room temperature and the supernatant was decanted. The remaining pale brown solid was washed with Et₂O and dried under vacuum to afford the title compound (17 mg, 33%): m.p. 297-300° C. ¹H-NMR (DMSO-d₆): δ 2.66 (s, 3H, CH₃), 2.67 (s, 3H, CH₃), 4.38 (s, 3H, N—CH₃), 7.35 (d, 1H, J=5.1 Hz, pyrimidine-H), 8.04 (t, 1H, J=5.5 Hz, pyridine-H), 8.57-8.59 (m, 2H, pyridine-H), 8.68 (d, 1H, J=5.4 Hz, pyrimidine-H), 9.44 (s, 1H, pyridine-H), 10.75 (s, 1H, NH). MS (DE MALDI-TOF) m/z 299.97 [M+H], C₁₅H₁₆N₅S requires 298.39.

### 5-[2-(6-chloro-pyridin-3-ylamino)-pyrimidin-4-yl]-3,4-dimethyl-3H-thiazol-2-one (4)

A mixture of 5-(3-dimethylamino-acryloyl)-3,4-dimethyl-3H-thiazol-2-one (120 mg, 5.0 mmol) and N-(6-chloro-pyridin-3-yl)-guanidine nitrate (170 mg, 7.0 mmol), prepared by guanylation of 6-chloro-pyridin-3-ylamine with aq cyanamide solution in the presence of HNO₃, in MeCN (5 mL) was treated with NaOH (0.60 g, 15 mmol). After heating at 160° C. for 20 min in a microwave reactor (Smith Creator, Personal Chemistry Ltd.), the solvent was evaporated and the residue was purified by SiO₂ gel chromatography (EtOAc/PE, 1:1). The title compound was obtained after recystallisation from MeOH (97 mg, 58%) as a grey solid. Anal. RP-HPLC: t_{R}=17.8 min (0-60% MeCN, purity >95%). ¹H-NMR (DMSO-d₆): δ2.61 (s, 3H, CH₃), 3.33 (s, 3H, CH₃), 7.09 (d, 1H, J=5.5 Hz, pyrimidine-H), 7.51 (d, 1H, J=8.5 Hz, pyridine-H), 8.27 (m, 1H, pyridine-H), 8.53 (d, 1H, J=5.5 Hz, pyrimidine-H), 8.81 (1H, d, J=2.75 Hz, pyridine-H). MS (ESI⁺) m/z 334.04 [M+H]⁺, C₁₄H₁₂N₅OSCl requires 333.80.

The following compounds were prepared in an analogous manner:

### (6-Chloro-pyridin-3-yl)-[4-(2,4-dimethyl-thiazol-5-yl)-pyrimidin-2-yl]-amine (3)

Yellow solid (29%). Anal. RP-HPLC: t_{R}=20.3 min (0-60% MeCN, purity >95%).

¹H-NMR (CD₃OD): δ 2.68 (s, 3H, CH₃), 2.69 (s, 3H, CH₃), 7.14 (d, 1H, J=5.0 Hz, pyrimidine-H), 7.38 (d, 1H, J=9.0 Hz, pyridyine-H), 8.24 (m, 1H, pyridine-H), 8.49 (d, 1H, J=5.0 Hz, pyrimidine-H), 8.78 (1H, d, J=2.5 Hz, pyridine-H).

[4-(2-Amino-4-methyl-thiazol-5-yl)-pyrimidin-2-yl]-(6-methoxy-pyridin-3-yl)-amine (5). Yellow solid. Anal. RP-HPLC: t_{R}=8.6 min (10-70% MeCN, purity >95%). ¹H-NMR (DMSO-d₆): δ 52.41 (s, 3H, CH₃), 3.80 (s, 3H, OCH₃), 6.75 (d, 1H, J=8.5 Hz, pyridine-H), 6.85 (d, 1H, J=5.5 Hz, pyrimidine-H), 7.49 (s, 2H, NH₂), 7.97 (m, 1H, pyridine-H), 8.28 (d, 1H, J=5.5 Hz, pyrimidine-H), 8.54 (sbr, 1H, pyridine-H), 9.32 (sbr, 1H, NH). MS (ESI⁺) m/z 315.14 [M+H]⁺, C₁₄H₁₄N₆OS requires 314.37.

### [4-(2,4-Dimethyl-thiazol-5-yl)-pyrimidin-2-yl]-(6-methoxy-pyridin-3-yl)-amine (6)

Light yellow solid. Anal. RP-HPLC: t_{R}=12.1 min (10-70% MeCN, purity >95%).

¹H-NMR (DMSO-d₆): δ 2.49 (s, 3H, CH₃), 2.63 (s, 3H, CH₃), 3.81 (s, 3H, OCH₃), 6.79 (d, 1H, J=8.0 Hz, pyridine-H), 7.05 (d, 1H, J=5.0 Hz, pyrimidine-H), 7.99 (m, 1H, pyridine-H), 8.48 (m, 2H, pyrimidine-H and pyridine-H), 9.56 (sbr, 1H, NH). MS (ESI⁺) m/z 314.01 [M+H]⁺, C₁₅H₁₅N₅OS requires 313.38.

### [4-(2-Amino-4-methyl-thiazol-5-yl)-pyrimidin-2-yl]-(6-chloro-pyridin-3-yl)-amine (7)

Brown solid. Anal. RP-HPLC: t_{R}=10.6 min (10-70% MeCN, purity >95%). ¹H-NMR (DMSO-d₆): δ 2.42 (s, 3H, CH₃), 6.95 (d, 1H, J=5.0 Hz, pyrimidine-H), 7.40 (d, 1H, J=9.0 Hz, pyridine-H), 7.55 (sbr, 2H, NH₂), 8.12 (m, 1H, pyridine-H), 8.36 (d, 1H, J=5.5 Hz, pyrimidine-H), 8.88 (m, 1H, pyridine-H), 9.77 (sbr, 1H, NH). MS (ESI⁺) m/z 319.00 [M+H]⁺, C₁₃H₁₁ClN₆S requires 318.79.

### (6-Methoxy-pyridin-3-yl)-[4-(4-methyl-2-methylamino-thiazol-5-yl)-pyrimidin-2-yl]-amine (8)

Brown solid. Anal. RP-HPLC: t_{R}=9.2 min (10-70% MeCN, purity >95%). ¹H-NMR (DMSO-d₆): δ 2.44 (s, 3H, CH₃), 3.23 (s, 3H, CH₃), 3.80 (s, 3H, OCH₃), 6.75 (d, 1H, J=8.5 Hz, pyridine-H), 6.86 (d, 1H, J=4.5 Hz, pyrimidine-H), 7.98 (m, 1H, pyridine-H), 8.04 (sbr, 1H, NH), 8.28 (d, 1H, J=5.5 Hz, pyrimidine-H), 8.50 (m, 1H, pyridine-H), 9.32 (sbr, 1H, NH). MS (ESI⁺) m/z 329.04 [M+H]⁺, C₁₅H₁₆N₆OS requires 328.39.

### (6-Chloro-pyridin-3-yl)-[4-(4-methyl-2-methylamino-thiazol-5-yl)-pyrimidin-2-yl]-amine (9)

Brown solid. Anal. RP-HPLC: t_{R}=11.3 min (10-70% MeCN, purity >95%). ¹H-NMR (DMSO-d₆): δ 2.43 (s, 3H, CH₃), 3.25 (s, 3H, CH₃), 6.97 (d, 1H, J=5.5 Hz, pyrimidine-H), 7.40 (d, 1H, J=9.0 Hz, pyridine-H), 8.37 (m, 1H, pyridine-H), 8.37 (m, 1H, pyrimidine-H), 8.84 (m, 1H, pyridine-H), 9.77 (sbr, 1H, NH). MS (ESI⁺) m/z 333.01 [M+H]⁺, C₁₄H₁₃ClN₆S requires 332.81.

### [4-(2-Dimethylamino-4-methyl-thiazol-5-yl)-pyrimidin-2-yl]-(6-methoxy-pyridin-3-yl)-amine (10)

Brown solid. Anal. RP-HPLC: t_{R}=10.1 min (10-70% MeCN, purity >95%). ¹H-NMR (DMSO-d₆): δ2.46 (s, 3H, CH₃), 3.08 (s, 3H, CH₃), 3.80 (s, 1H, OCH₃), 6.78 (d, 1H, J=9.0 Hz, pyridine-H), 6.88 (d, 1H, J=5.0 Hz, pyrimidine-H), 8.04 (d, 1H, J=9.0 Hz, pyridine-H), 8.29 (d, 1H, J=5.0 Hz, pyrimidine-H), 8.44 (m, 1H, pyridine-H), 9.33 (sbr, 1H, NH). MS (ESI⁺) m/z 343.14 [M+H]⁺, C₁₆H₁₈N₆OS requires 342.42.

### 3-Ethyl-5-[2-(6-methoxy-pyridin-3-ylamino)-pyrimidin-4-yl]-4-methyl-3H-thiazol-2-one (11)

Brown Solid (39%). Anal. RP-HPLC: t_{R}=15.6 min (0-60% MeCN, purity >95%).

¹H-NMR (DMSO-d₆): δ1.15 (t, 3H, J=6.83 Hz, CH₃), 2.55 (s, 1H, CH₃), 3.81 (s, 3H, OCH₃), 3.26 (m, 2H, CH₂), 6.79 (d, 1H, J=9.3 Hz, pyridine-H), 6.91 (d, 1H, J=5.4 Hz, pyrimidine-H), 7.97 (dd, 1H, J=9.28, 2.93 Hz, pyridine-H), 8.38 (d, 1H, J=5.37 Hz, pyrimidine-H), 8.44 (d, 1H, J=2.93, pyridine-H), 9.50 (s, 1H, NH). ¹³C-NMR (DMSO-d₆): δ14.5, 14.7, 37.3, 53.7, 109.0, 110.3, 128.4, 131.82, 132.4, 137.8, 138.4, 152.7, 159.5, 160.3, 164.7, 170.1. MS (ESI⁺) m/z 342.09 [M+H]⁺, C₁₆H₁₇N₅O₂S requires 343.40.

### [4-(2-Ethylamino-4-methyl-thiazol-5-yl)-pyrimidin-2-yl]-(6-methoxy-pyridin-3-yl)-amine (12)

Yellow Solid. Anal. RP-HPLC: t_{R}=10.1 min (10-70% MeCN, purity 100%). ¹H-NMR (DMSO-d₆), 1.16 (m, 3H, CH₃), 2.48 (s, 1H, CH₃), 3.26 (m, 2H, CH₂), 3.80 (s, 3H, OCH₃), 6.76 (d, 1H, J=8.0 Hz, pyridyl-H), 6.86 (d, 1H, J=5.5 Hz, pyrimidinyl-H), 7.98 (m, 1H, pyridyl-H), 8.09 (t, 1H, J=5.5 Hz, pyridyl-H), 8.28 (d, 1H, J=5.5 Hz, pyrimidinyl-H), 8.50 (s, 1H, NH), 9.32 (s, 1H, NH). MS (ESI⁺) m/z 343.25 [M+H]⁺, C I₆H 18N₆OS requires 342.42.

### {4-[2-(2-Methoxy-ethylamino)-4-methyl-thiazol-5-yl]-pyrimidin-2-yl}-(6-methoxy-pyridin-3-yl)-amine (13)

Yellow Solid. Anal. RP-HPLC: t_{R}=18.1 min (10-70% MeCN, purity >95%). ¹H-NMR (DMSO-d₆) δ: 2.43 (s, 3H, CH₃), 3.23 (t, 2H, J=7.2 Hz, CH₂), 3.41 (t, 2H, J=7.2 Hz, CH₂), 3.46 (s, 3H, OCH₃), 3.80 (s, 3H, OCH₃), 6.75 (d, 1H, J=8.8 Hz, pyridyl-H), 6.87 (d, 1H, J=5.4 Hz, pyrimidinyl-H), 7.99 (dd, 1H, J=2.9, 8.8 Hz, pyridyl-H), 8.18 (s, 1H, NH), 8.29 (d, 1H, J=5.4 Hz, pyrimidinyl-H), 8.51 (d, 1H, J=2.9 Hz, pyridyl-H), 9.33 (s, 1H, NH). MS (ESI⁺) m/z 373.75 [M+H]⁺, C₁₇H₂₀N₆O₂S requires 372.45.

### [4-(2,4-Dimethyl-thiazol-5-yl)-pyrimidin-2-yl]-(6-pyrrolidin-1-yl-pyridin-3-yl)-amine (14)

Yellow solid. Anal. RP-HPLC: t_{R}=16.5 min (0-60% MeCN, purity 100%). ¹H-NMR (CDCl₃) δ: 2.01 (m, 4H, CH₂), 2.66 (s, 3H, CH₃), 2.71 (s, 3H, CH₃), 3.47 (m, 3H, CH₃), 6.40 (d, 1H, J=9.0 Hz, pyridyl-H), 6.84 (d, 1H, J=5.5 Hz, pyrimidinyl-H), 7.78 (d, 1H, J=9.0 Hz, pyridyl-H), 8.24 (d, 1H, J=3.0, pyridyl-H), 8.34 (d, 1H, J=5.0 Hz, pyrimidinyl-H). MS (ESI⁺) m/z 353.32 [M+H]⁺, C₁₈H₂₀N₆S requires 352.46.

### [4-(4-Methyl-2-methylamino-thiazol-5-yl)-pyrimidin-2-yl]-[6-(4-methyl-piperazin-1-yl)-pyridin-3-yl]-amine (15)

Yellow Solid. Anal. RP-HPLC: t_{R}=7.1 min (10-70% MeCN, purity 100%). ¹H-NMR (DMSO-d₆) δ: 2.21 (s, 3H, CH₃), 2.40 (m, 4H, CH₂), 2.45 (s, 3H, CH₃), 2.84 (sbr, 3H, CH₃), 3.36 (m, 4H, CH₂), 6.79 (d, 1H, J=8.5 Hz, pyridyl-H), 6.82 (d, 1H, J=5.5 Hz, pyrimidinyl-H), 7.86 (dd, 1H, J=2.0, 8.5 Hz, pyridyl-H), 8.02 (m, 1H, NH), 8.26 (d, 1H, J=5.0 Hz, pyrimidinyl-H), 8.45 (d, 1H, J=2.5 Hz, pyridyl-H), 9.16 (s, 1H, NH). MS (ESI⁺) m/z 397.36 [M+H]⁺, C₁₉H₂₄N₈S requires 396.51.

### (6-Methoxy-pyridin-3-yl)-[4-(4-methyl-2-morpholin-4-yl-thiazol-5-yl)-pyrimidin-2-yl]-amine (16)

Yellow Solid. Anal. RP-HPLC: t_{R}=11.5 min (10-70% MeCN, purity >95%). ¹H-NMR (DMSO-d₆) d: 2.48 (s, 3H, CH₃), 3.46 (t, 4H, J=5.0 Hz, CH₂), 3.71 (t, 4H, J=5.0 Hz, CH₂), 3.81 (s, 3H, OCH₃), 6.80 (d, 1H, J=9.0 Hz, pyridyl-H), 6.91 (d, 1H, J=5.0 Hz, pyrimidinyl-H), 8.04 (dd, 1H, J=3.0, 9.0 Hz, pyridyl-H), 8.33 (d, 1H, J=5.5 Hz, pyrimidinyl-H), 8.43 (d, 1H, J=2.5 Hz, pyridyl-H), 9.39 (s, 1H, NH). MS (ESI⁺) m/z 385.48 [M+H]⁺, C₁₈H₂₀N₆O₂S requires 384.46.

Kinase Assays

The compounds from the examples above were investigated for their ability to inhibit the enzymatic activity of various protein kinases. This was achieved by measurement of incorporation of radioactive phosphate from ATP into appropriate polypeptide substrates. Recombinant protein kinases and kinase complexes were produced or obtained commercially. Assays were performed using 96-well plates and appropriate assay buffers (typically 25 mM β-glycerophosphate, 20 mM MOPS, 5 mM EGTA, 1 mM DTT, 1 mM Na₃VO₃, pH 7.4), into which were added 2-4 μg of active enzyme with appropriate substrates. The reactions were initiated by addition of Mg/ATP mix (15 mM MgCl₂+100 μM ATP with 30-50 kBq per well of [γ-³²P]-ATP) and mixtures incubated as required at 30° C. Reactions were stopped on ice, followed by filtration through p81 filterplates or GF/C filterplates (Whatman Polyfiltronics, Kent, UK). After washing 3 times with 75 mM aq orthophosphoric acid, plates were dried, scintillant added and incorporated radioactivity measured in a scintillation counter (TopCount, Packard Instruments, Pangboume, Berks, UK). Compounds for kinase assay were made up as 10 mM stocks in DMSO and diluted into 10% DMSO in assay buffer. Data was analysed using curve-fitting software (GraphPad Prism version 3.00 for Windows, GraphPad Software, San Diego Calif. USA) to determine IC₅₀ values (concentration of test compound which inhibits kinase activity by 50%.). The IC₅₀ values of selected compounds of the invention are shown in Table 1.

MTT Cytotoxicity Assay

The compounds from the examples above were subjected to a standard cellular proliferation assay using human tumour cell lines obtained from the ATCC (American Type Culture Collection, 10801 University Boulevard, Manessas, Va. 20110-2209, USA). Standard 72-h MTT (thiazolyl blue; 3-[4,5-dimethylthiazol-2-yl]-2,5-diphenyltetrazolium bromide) assays were performed [67, 68]. In short: cells were seeded into 96-well plates according to doubling time and incubated overnight at 37° C. Test compounds were made up in DMSO and a 1/3 dilution series prepared in 100 μL cell media, added to cells (in triplicates) and incubated for 72 ho at 37° C. MTT was made up as a stock of 5 mg/mL in cell media and filter-sterilised. Media was removed from cells followed by a wash with 200 μL PBS. MTT solution was then added at 20 μL per well and incubated in the dark at 37° C. for 4 h. MTT solution was removed and cells again washed with 200 μL PBS. MTT dye was solubilised with 200 μL per well of DMSO with agitation. Absorbance was read at 540 nm and data analysed using curve-fitting software (GraphPad Prism version 3.00 for Windows, GraphPad Software, San Diego Calif. USA) to determine IC₅₀ values (concentration of test compound which inhibits cell growth by 50%). The IC₅₀ values of selected compounds of the invention are shown in Table 2. Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Structures of example compounds and inhibitory activity against various protein kinases.

| | | | Kinase inhibition Kᵢ (μM) | | | | | | | |
|---|---|---|---|---|---|---|---|---|---|---|
| | | | | CDK2/ | | CDK4/ | CDK7/ | CDK9/ | | |
| | | | CDK1/ | cyclin | CDK2/ | cyclin | cyclin | cyclin | | Aurora- |
| No. | Structure | Name | cyclin B | A | cyclin E | D1 | H | T1 | GSK3β | A |
| 1 | 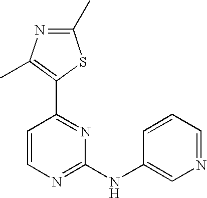 | [4-(2,4-Dimethyl- thiazol-5-yl)- pyrimidin-2-yl]- pyridin-3-yl-amine | | | 0.11 | 0.34 | | 0.08 | | |
| 2 | 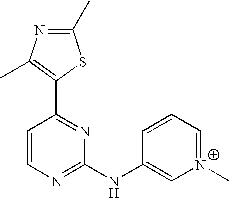 | 3-[4-(2,4-Dimethyl- thiazol-5-yl)- pyrimidin-2-ylamino]- 1-methyl-pyridinium | | | 1.5 | 4.1 | | | | |
| 3 | 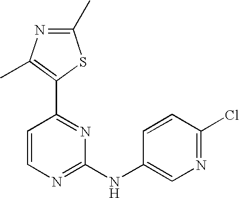 | (6-Chloro-pyridin-3- yl)-[4-(2,4-dimethyl- thiazol-5-yl)- pyrimidin-2-yl]-amine | 4.2 | 1.7 | 0.35 | 0.66 | 7.2 | 0.97 | 0.11 | 0.86 |
| 4 | 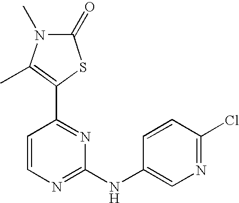 | 5-[2-(6-chloro-pyridin- 3-ylamino)-pyrimidin- 4-yl]-3,4-dimethyl- 3H-thiazol-2-one | 1.5 | | 0.14 | | | 0.021 | 0.011 | |
| 5 | 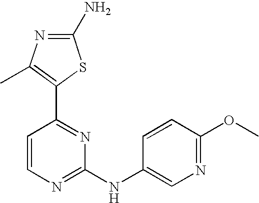 | [4-(2-Amino-4-methyl- thiazol-5-yl)- pyrimidin-2-yl]-(6- methoxy-pyridin-3- yl)-amine | 5.5 | 0.95 | 0.07 | 0.06 | 0.71 | 0.005 | 0.35 | |
| 6 | 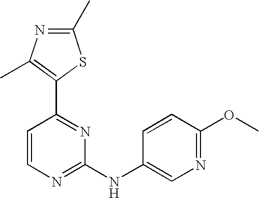 | [4-(2,4-Dimethyl- thiazol-5-yl)- pyrimidin-2-yl]-(6- methoxy-pyridin-3- yl)-amine | 2.8 | 1.4 | 0.13 | 0.46 | 1.4 | 0.28 | | |
| 7 | 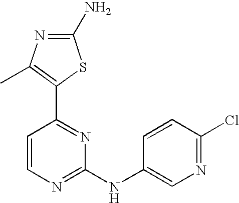 | [4-(2-Amino-4-methyl- thiazol-5-yl)- pyrimidin-2-yl]-(6- chloro-pyridin-3-yl)- amine | 2.0 | 0.86 | 0.05 | 0.07 | 1.5 | 0.005 | 0.16 | |
| 8 | 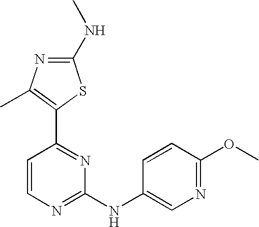 | (6-Methoxy-pyridin-3- yl)-[4-(4-methyl-2- methylamino-thiazol- 5-yl)-pyrimidin-2-yl]- amine | 0.84 | 0.27 | 0.03 | 0.04 | 0.43 | 0.006 | 0.05 | |
| 9 | 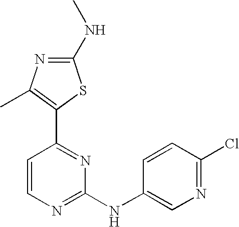 | (6-Chloro-pyridin-3- yl)-[4-(4-methyl-2- methylamino-thiazol- 5-yl)-pyrimidin-2-yl]- amine | | 0.48 | 0.08 | 0.98 | | 0.11 | | 1.0 |
| 10 | 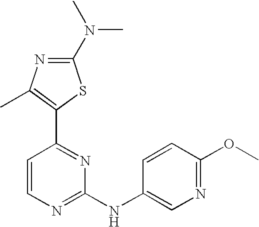 | [4-(2-Dimethylamino- 4-methyl-thiazol-5-yl)- pyrimidin-2-yl]-(6- methoxy-pyridin-3-yl)- amine | 1.2 | 0.33 | 0.12 | 0.07 | 2.3 | 0.14 | 0.11 | |
| 11 | 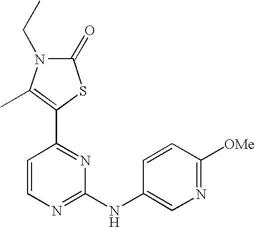 | 3-Ethyl-5-[2-(6- methoxy-pyridin-3- ylamino)-pyrimidin-4- yl]-4-methyl-3H- thiazol-2-one | 3.4 | 0.37 | 0.11 | 1.2 | 0.23 | 0.027 | 0.013 | |
| 12 | 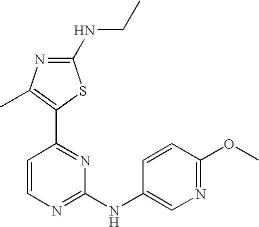 | [4-(2-Ethylamino-4- methyl-thiazol-5-yl)- pyrimidin-2-yl]-(6- methoxy-pyridin-3-yl)- amine | 0.10 | 0.03 | 0.05 | 0.08 | 0.13 | 0.09 | 0.06 | |
| 13 | 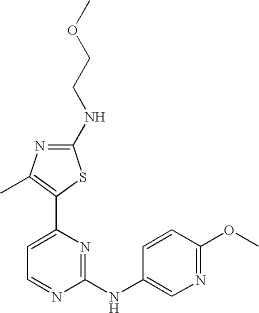 | {4-[2-(2-Methoxy- ethylamino)-4-methyl- thiazol-5-yl]- pyrimidin-2-yl}-(6- methoxy-pyridin-3- yl)-amine | 2.3 | 0.95 | 0.25 | 0.25 | 0.65 | 0.12 | 0.10 | |
| 14 | 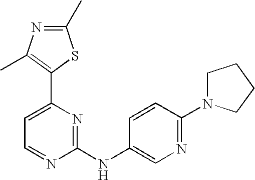 | [4-(2,4-Dimethyl- thiazol-5-yl)- pyrimidin-2-yl]-(6- pyrrolidin-1-yl- pyridin-3-yl)-amine | 2.1 | 1.4 | | 2.6 | 0.73 | 0.10 | | 0.12 |
| 15 | 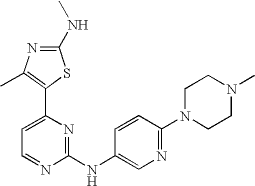 | [4-(4-Methyl-2- methylamino-thiazol- 5-yl)-pyrimidin-2-yl]- [6-(4-methyl- piperazin-1-yl)- pyridin-3-yl]-amine | | 1.5 | | 0.08 | 0.28 | 0.08 | 2.9 | |
| 16 | 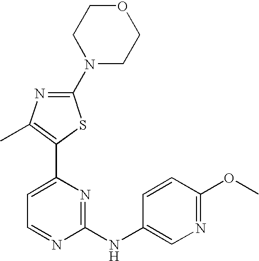 | (6-Methoxy-pyridin-3- yl)-[4-(4-methyl-2- morpholin-4-yl- thiazol-5-yl)- pyrimidin-2-yl]-amine | | | | | | | | |

TABLE 2
Anti-proliferative activity of example compounds against transformed
human cell lines in vitro.

| | |
|---|---|
| Com- | 72-h MTT IC₅₀ (μM) |

| pound | Cell line | | | |
|---|---|---|---|---|
| No. | A549 | HT29 | Saos-2 | Average |
| 1 | 3.2 | 3.7 | 3.7 | 3.6 ± 0.3 |
| 2 | 11.6 | 86.0 | 48.5 | 48.7 ± 37.2 |
| 3 | 9.8 | 2.8 | 4.4 | 5.7 ± 3.7 |
| 4 | 1.1 | 0.6 | 1.7 | 1.1 ± 0.6 |
| 5 | 3.5 | 4.0 | 3.1 | 3.5 ± 0.4 |
| 6 | 5.1 | 1.4 | 7.5 | 4.6 ± 3.1 |
| 7 | 3.3 | 3.4 | 3.1 | 3.2 ± 0.1 |
| 8 | 1.4 | 2.0 | 1.7 | 1.7 ± 0.3 |
| 9 | 3.7 | 4.7 | 5.2 | 4.6 ± 0.8 |
| 10 | 2.4 | 1.7 | 1.8 | 2.0 ± 0.4 |
| 12 | 1.2 | 0.8 | 1.5 | 1.2 ± 0.4 |
| 14 | 0.3 | | 0.32 | 0.3 ± 0.0 |

### REFERENCES

1. Manning, G.; Whyte, D. B.; Martinez, R.; Hunter, T.; Sudarsanam, S. The protein kinase complement of the human genome. Science 2002, 298, 1912-1934.
2. Kostich, M.; English, J.; Madison, V.; Gheyas, F.; Wang, L. et al. Human members of the eukaryotic protein kinase family. Genome Biology 2002, 3, research0043.0041-0043.0012.
3. Dancey, J.; Sausville, E. A. Issues and progress with protein kinase inhibitors for cancer treatment. Nat. Rev. Drug Disc. 2003, 2, 296-313.
4. Cockerill, G. S.; Lackey, K. E. Small molecule inhibitors of the class 1 receptor tyrosine kinase family. Current Topics in Medicinal Chemistry 2002, 2, 1001-1010.
5. Fabbro, D.; Ruetz, S.; Buchdunger, E.; Cowan-Jacob, S. W.; Fendrich, G. et al. Protein kinases as targets for anticancer agents: from inhibitors to useful drugs. Pharmacol. Ther. 2002, 93, 79-98.
6. Cohen, P. Protein kinases—the major drug targets of the twenty-first century? Nat. Rev. Drug Disc. 2002, 1, 309-315.
7. Bridges, A. J. Chemical inhibitors of protein kinases. Chem. Rev. 2001, 101(8), 2541-2571.
8. Wang, S.; Meades, C.; Wood, G.; Osnowski, A.; Fischer, P. M. N-(4-(4-methylthiazol-5-yl) pyrimidin-2-yl)-N-phenylamines as antiproliferative compounds. PCT Intl. Patent Appl. Publ. WO 2003029248; Cyclacel Limited, UK.
9. Wu, S. Y.; McNae, I.; Kontopidis, G.; McClue, S. J.; Mclnnes, C. et al. Discovery of a Novel Family of CDK Inhibitors with the Program LIDAEUS: Structural Basis for Ligand-Induced Disordering of the Activation Loop. Structure 2003, 11, 399-410.
10. Fischer, P. M.; Wang, S.; Wood, G. Inhibitors of cyclin dependent kinases as anti-cancer agent. PCT Intl. Patent Appl. Publ. WO 02/079193; Cyclacel Limited, UK,.
11. Wang, S.; Fischer, P. M. Anti-cancer compounds. US Patent Appl. Publ. 2002/0019404.
12. Fischer, P. M.; Wang, S. 2-substituted 4-heteroaryl-pyrimidines and their use in the treatmetn of proliferative disorders. PCT Intl. Patent Appl. Publ. WO 2001072745; Cyclacel Limited, UK.
13. Knockaert, M.; Greengard, P.; Meijer, L. Pharmacological inhibitors of cyclin-dependent kinases. Trends Pharmacol. Sci. 2002, 23, 417-425.
14. Fischer, P. M.; Endicott, J.; Meijer, L. Cyclin-dependent kinase inhibitors. Progress in Cell Cycle Research; Editions de la Station Biologique de Roscoff: Roscoff, France, 2003; pp 235-248.
15. Fravolini, A.; Grandolini, G.; Martani, A. New heterocyclic ring systems from α-hydroxymethylene ketones. V. Reaction of 2-methyl-6-hydroxymethylene-4,5,6,7-tetrahydrobenzothiazol-7-one with amines and amidines. Gazz. Chim. Ital. 1973, 103, 1063-1071.
16. Cleaver, L.; Croft, J. A.; Ritchie, E.; Taylor, W. C. Chemical studies of the Proteaceae. IX. Synthesis of 5-alkylresorcinols from aliphatic precursors. Aust. J. Chem. 1976, 29, 1989-2001.
17. Fadda, A. A.; El-Houssini, M. S. Synthesis of cyclic ketones by activated nitriles. J. Ind. Chem. Soc. 1990, 67,915-917.
18. Kost, A. N.; Ovseneva, L. G. Synthesis of 4-substituted dihydroresorcinols. Zh. Obshch. Khim 1962, 32, 3983-3986.
19. Lehmann, G.; Luecke, B.; Schick, H.; Hilgetag, G. 2-Substituted 7-oxo-4,5,6,7-tetrahydrobenzothiazoles. Z. Chem. 1967, 7, 422.
20. Bell, R. P.; Davis, G. G. Kinetics of the bromination of some enols and their anions. J. Chem. Soc 1965, 353-361.
21. Fravolini, A.; Grandolini, G.; Martani, A. New heterocyclic ring systems from α-hydroxymethylene ketones. III. Pyrazolobenzothiazoles and thiazolo-benzoisoxazoles. Gazz. Chim. Ital. 1973, 103, 755-769.
22. Bredereck, H.; Effenberger, F.; Botsch, H. Acid amide reactions. XLV. Reactivity of formamidines, dimethylformamide diethyl acetal (amide acetal), and bis(dimethylamino)methoxymethane (aminal ester). Chem. Ber. 1964, 97, 3397-3406.
23. Wang D, De la Fuente C, Deng L, Wang L, Zilberman I, Eadie C, Healey M, Stein D, Denny T, Harrison LE, Meijer L, Kashanchi F. Inhibition of human immunodeficiency virus type I transcription by chemical cyclin-dependent kinase inhibitors. J. Virol. 2001; 75: 7266-7279.
24. Chen, Y. H.; Hansen, L.; Chen, M. X.; Bjorbaek, C.; Vestergaard, H.; Hansen, T.; Cohen, P. T.; Pedersen, O. Diabetes, 1994, 43, 1234.
25. Nikoulina, S. E.; Ciaraldi, T. P.; Mudaliar, S.; Mohideen, P.; Carter, L.; Henry, R. R. Diabetes, 2000, 49, 263.
26. Goedert, M. Curr. Opin. Gen. Dev., 2001, 11, 343.
27. Mattson, M. P. Nat. Rev. Mol. Cell. Biol., 2000, 1, 120.
28. Zhu, A. J.; Watt, F. M. Development, 1999, 126, 2285.
29. DasGupta, R.; Fuchs, E. Development, 1999, 126, 4557.
30. Sunkel et al., J. Cell Sci., 1988, 89, 25.
31. Llamazares et al., Genes Dev., 1991, 5, 2153.
32. Glover et al., Genes Dev., 1998, 12, 3777.
33. Lee et al., Proc. Natl. Acad. Sci. USA, 1998, 95, 9301.
34. Leung et al., Nat. Struct. Biol., 2002, 9, 719.
35. Kauselmann et al., EMBO J., 1999, 18, 5528.
36. Nigg, Curr. Opin. Cell Biol., 1998, 10, 776.
37. Yuan et al., Cancer Res., 2002, 62, 4186.
38. Seong et al., J. Biol. Chem., 2002, 277, 32282.
39. Lane et al., J. Cell. Biol., 1996, 135, 1701.
40. Cogswell et al., Cell Growth Differ., 2000, 11, 615.
41. Liu et al., Proc. Natl. Acad. Sci. USA, 2002, 99, 8672.
42. Toyoshima-Morimoto et al., Nature, 2001, 410, 215.
43. Roshak et al., Cell. Signalling, 2000, 12, 405.
44. Smits et al., Nat. Cell Biol., 2000, 2, 672.
45. van Vugt et al., J. Biol. Chem., 2001, 276, 41656.
46. Sumara et al., Mol. Cell, 2002, 9, 515.
47. Golan et al., J. Biol. Chem., 2002, 277, 15552.
48. Kotani et al., Mol. Cell, 1998, 1, 371.
49. Feng et al., Cell Growth Differ., 2001, 12, 29.
50. Dai et al., Oncogene, 2002, 21, 6195.
51. Nurse, Nature, 1990, 344, 503.
52. Nigg, Nat. Rev. Mol. Cell Biol., 2001, 2, 21.
53. Hagting et al., EMBO J., 1998, 17, 4127.
54. Hagting et al., Curr. Biol., 1999, 9, 680.
55. Yang et al., J. Biol. Chem., 2001, 276, 3604.
56. Takizawa et al., Curr. Opin. Cell Biol., 2000, 12, 658.
57. Seki et al., Mol. Biol. Cell, 1992, 3, 1373.
58. Heald et al., Cell, 1993, 74, 463.
59. Dalal et al., Mol. Cell. Biol., 1999, 19, 4465.
60. Toyoshima-Morimoto et al., Nature, 2001, 410, 215.
61. Toyoshima-Morimoto et al., EMBO Rep., 2002, 3, 341.
62. Wang et al., Mol. Cell. Biol., 2002, 22, 3450.
63. Tyrrell, E.; Brookes, P. Synthesis 2003, 469-483.
64. Rocca, P.; Cochennec, C.; Marsais, F.; Thomas-dit-Dumont, L.; Mallet, M. et al. J. Org. Chem. 1993, 58, 7832-7838.
65. Bredereck, H.; Effenberger, F.; Botsch, H. Chem. Ber. 1964, 97, 3397-3406.
66. Zimmermann, J.; Caravatti, G.; Mett, H.; Meyer, T.; Müller, M. et al. Arch. Pharm. Pharm. Med. Chem. 1996, 329, 371-376.
67. Haselsberger, K.; Peterson, D. C.; Thomas, D. G.; Darling, J. L. Anti Cancer Drugs 1996, 7, 331-8.
68. Loveland, B. E.; Johns, T. G.; Mackay, I. R.; Vaillant, F.; Wang, Z. X.; Hertzog, P. J. Biochemistry International 1992, 27, 501-10.

1. A compound of formula I, or a pharmaceutically acceptable salt thereof, 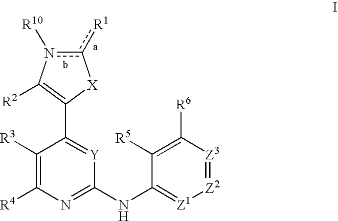
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups;
where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine.

2. A compound of formula Ib, or a pharmaceutically acceptable salt thereof, 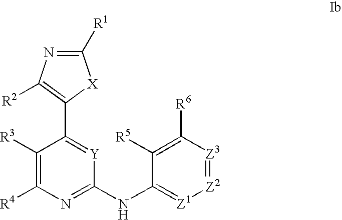
wherein
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups;
where the compound is other than [4-(2,4-dimethyl-thiazol-5-yl)-pyrimidin-2-yl]-pyridin-2-yl-amine and 4-[4-fluorophenyl)-1-(1-methyl-4-piperidinyl)-1H-imidazol-5-yl]-N-4-pyridinyl-2-pyrimidinamine.

3. The compound according to claim 1 or claim 2, wherein each R⁷ is independently H, halogen, NR^{b}R^{c}, OR^{d} or a saturated or unsaturated group containing between 1 and 20 C atoms, optionally containing one or more heteroatoms selected from from N, S, and O, and optionally substituted with one or more R⁹ groups.

4. The compound according to claim 1 or claim 2, wherein each R⁷ is independently H, halogen, NR^{b}R^{c}, OR^{d} or is an alicyclic, alkyl, cycloalkyl, aryl or aralkyl group, each of which optionally contain one to six heteroatoms selected from N, S and O, and each of which is optionally substituted by one to six R⁹ groups.

5. The compound according to claim 1 or claim 2, wherein each R⁷ is independently selected from H, OR^{d}, NR^{b}R^{c}, halogen and an alicyclic group optionally comprising one or more heteroatoms and which is optionally substituted by one or more R⁹ groups.

6. The compound according to claim 1 or claim 2, wherein each R⁷ is independently selected from H, OR^{d}, NR^{b}R^{c}, halogen and an alicyclic group selected from pyrrolidinyl, piperidinyl, morpholino and piperazinyl, each of which is optionally substituted by one or more R⁹ groups.

7. The compound according to claim 1 or claim 2, wherein each R⁷ is independently selected from Me, Cl, OMe, OEt, NH₂, NHMe, NHEt, NMe₂, N-pyrrolidinyl, N-piperidinyl, N-morpholino and N-piperazinyl.

8. The compound according to claim 1 or claim 2, wherein R^{a-q} are each independently H, Me or Et, said Me or Et groups being optionally substituted by one or more R⁹ groups.

9. The compound according to claim 1 or claim 2, wherein R⁹ is selected from H, halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

10. The compound according to claim 1 or claim 2, wherein R⁹ is selected from OMe, halogen, NH₂, CN, NO₂, CF₃, OEt, NMe₂, NHMe and OH.

11. The compound according to claim 1 or claim 2, wherein Z² is N or NR^{a+} and Z¹ and Z³ are each independently CR⁷.

12. The compound according to claim 1 or claim 2, wherein Z² is N or NR^{a+}, Z¹ is C—H and Z³ is C—Cl or C—OMe.

13. The compound according to claim 1 or claim 2, wherein Y is N.

14. The compound according to claim 1 or claim 2, wherein X is S.

15. The compound according to claim 1 or claim 2, wherein R¹ is selected from Me, OMe, OEt, NH₂, NHMe, NHEt and NMe₂.

16. The compound according to claim 1 or claim 2, wherein R² is Me.

17. The compound according to claim 1 or claim 2, wherein R³, R⁴, R⁵ and R⁶ are all H.

18. The compound according to claim 1 of formula Id, or a pharmaceutically acceptable salt thereof, 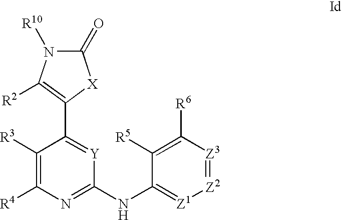
wherein R²⁻⁶, R¹⁰, X, Y, Z¹, Z² and Z³ are as defined in any one of claims 1, or 3 to 17.

19. A compound according to claim 1 which is selected from the following:
[4-(2,4-Dimethyl-thiazol-5-yl)-pyrimidin-2-yl]-pyridin-3-yl-amine [1];
3-[4-(2,4-Dimethyl-thiazol-5-yl)-pyrimidin-2-ylamino]-1-methyl-pyridinium [2];
(6-Chloro-pyridin-3-yl)-[4-(2,4-dimethyl-thiazol-5-yl)-pyrimidin-2-yl]-amine [3];
5-[2-(6-chloro-pyridin-3-ylamino)-pyrimidin-4-yl]-3,4-dimethyl-3H-thiazol-2-one [4];
[4-(2-Amino-4-methyl-thiazol-5-yl)-pyrimidin-2-yl]-(6-methoxy-pyridin-3-yl)-amine [5];
[4-(2,4-Dimethyl-thiazol-5-yl)-pyrimidin-2-yl]-(6-methoxy-pyridin-3-yl)-amine [6];
[4-(2-Amino-4-methyl-thiazol-5-yl)-pyrimidin-2-yl]-(6-chloro-pyridin-3-yl)-amine [7];
(6-Methoxy-pyridin-3-yl)-[4-(4-methyl-2-methylamino-thiazol-5-yl)-pyrimidin-2-yl]-amine [8];
(6-Chloro-pyridin-3-yl)-[4-(4-methyl-2-methylamino-thiazol-5-yl)-pyrimidin-2-yl]-amine [9];
[4-(2-Dimethylamino-4-methyl-thiazol-5-yl)-pyy-midin-2-yl]-(6-methoxy-pyridin-3-yl)-amine [10];
3-Ethyl-5-[2-(6-methoxy-pyridin-3-ylamino)-pyrimidin-4-yl]-4-methyl-3H-thiazol-2-one [11];
[4-(2-Ethylamino-4-methyl-thiazol-5-yl)-pyrimidin-2-yl]-(6-methoxy-pyridin-3-yl)-amine [12];
{4-[2-(2-Methoxy-ethylamino)-4-methyl-thiazol-5-yl]-pyrimidin-2-yl}-(6-methoxy-pyridin-3-yl)-amine [13];
[4-(2,4-Dimethyl-thiazol-5-yl)-pyrimidin-2-yl]-(6-pyrrolidin-1-yl-pyridin-3-yl)-amine [14];
[4-(4-Methyl-2-methylamino-thiazol-5-yl)-pyrimidin-2-yl]-[6-(4-methyl-piperazin-1-yl)-pyridin-3-yl]-amine [15]; and
(6-Methoxy-pyridin-3-yl)-[4-(4-methyl-2-morpholin-4-yl-thiazol-5-yl)-pyrimidin-2-yl]-amine [16].

20. The compound according to claim 19 which is selected from the following: [1], [5], [7], [8], [9], [10], [12] and [15].

21. The compound according to claim 19 which is selected from the following: [8], [10], [12] and [14].

22. The pharmaceutical composition comprising a compound of claim 1 or claim 2, admixed with a pharmaceutically acceptable diluent, excipient or carrier.

23. A method of treating a proliferative disorder, said method comprising administering to a subject in need thereof, a compound of formula 1a, or a pharmaceutically acceptable salt thereof, in an amount sufficient to treat the proliferative disorder, wherein said compound of formula 1a is: 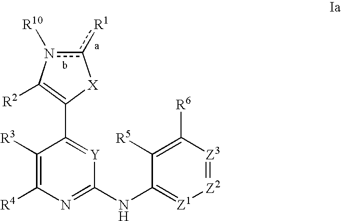
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

24. The method according to claim 23, wherein the proliferative disorder is cancer or leukemia.

25. The method according to claim 23, wherein the proliferative disorder is glomerulonephritis, rheumatoid arthritis, psoriasis or chronic obstructive pulmonary disorder.

26. A method of treating a viral disorder, said method comprising administering to a subject in need thereof, a compound of formula 1a, or a pharmaceutically acceptable salt thereof, in an amount sufficient to treat the viral disorder, wherein said compound of formula 1a is: 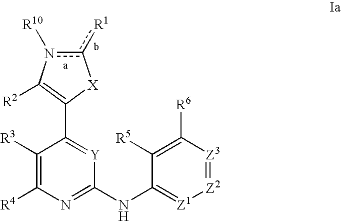
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

27. The method according to claim 26, wherein the viral disorder is selected from human cytomegalovirus (HCMV), herpes simplex virus type 1 (HSV-1), human immunodeficiency virus type I (HIV-1), and varicella zoster virus (VZV).

28. A method of treating a CNS disorder, said method comprising administering to a subject in need thereof, a compound of formula 1a, or a pharmaceutically acceptable salt thereof, in an amount sufficient to treat the CNS disorder, wherein said compound of formula 1a is: 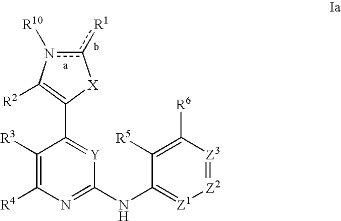
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

29. The method according to claim 28, wherein the CNS disorder is Alzheimer's disease or bipolar disorder.

30. A method of treating alopecia, said method comprising administering to a subject in need thereof, a compound of formula 1a, or a pharmaceutically acceptable salt thereof, in an amount sufficient to treat alopecia, wherein said compound of formula 1a is: 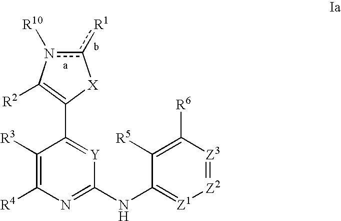
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

31. A method of treating a stroke, said method comprising administering to a subject in need thereof, a compound of formula 1a, or a pharmaceutically acceptable salt thereof, in an amount sufficient to treat the stroke, wherein said compound of formula 1a is: 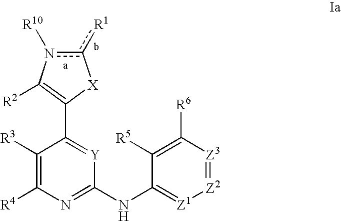
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

32. The method according to claim 23, wherein the compound of formula I is administered in an amount sufficient to inhibit at least one PLK enzyme.

33. The method according to claim 32, wherein the PLK enzyme is PLK1.

34. The method according to claim 23, wherein the compound of formula I is administered in an amount sufficient to inhibit at least one CDK enzyme.

35. The method according to claim 34, wherein the CDK enzyme is CDK1, CDK2, CDK3, CDK4, CDK6, CDK7, CDK8 and/or CDK9.

36. The method according to claim 23, wherein the compound of formula I is administered in an amount sufficient to inhibit aurora kinase.

37. A method of treating diabetes, said method comprising administering to a subject in need thereof, a compound of formula 1a, or a pharmaceutically acceptable salt thereof, in an amount sufficient to treat diabetes, wherein said compound of formula 1a is: 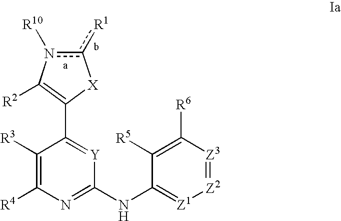
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

38. The method according to claim 37, wherein the diabetes is Type II diabetes.

39. The method according to claim 37, wherein the compound of formula I is administered in an amount sufficient to inhibit GSK.

40. The method according to claim 39, wherein the compound of formula I is administered in an amount sufficient to inhibit GSK3β.

41. Use of a compound of formula Ia in an assay for identifying further candidate compounds capable of inhibiting one or more of a cyclin dependent kinase, aurora kinase, GSK and a PLK enzyme, wherein said compound of formula 1a is: 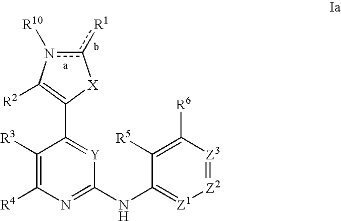
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

42. Use according to claim 41 wherein said assay is a competitive binding assay.

43. A process for preparing a compound of formula Ib, said process comprising the steps of: 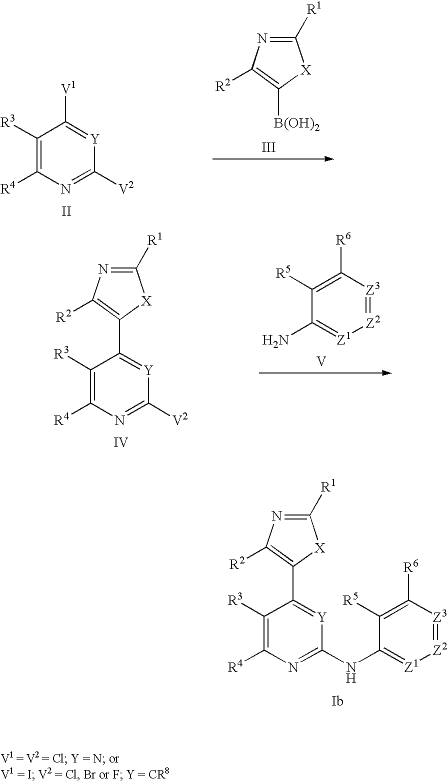
(i) reacting a heteroaryl boronic acid of formula III with a 2,4-dihalogenated pyrimidine or pyridine of formula II to form a compound of formula IV;
(ii) reacting said compound of formula IV with an aniline of formula V to form a compound of formula Ib.

44. A process for preparing a compound of formula Ib, said process comprising the steps of: 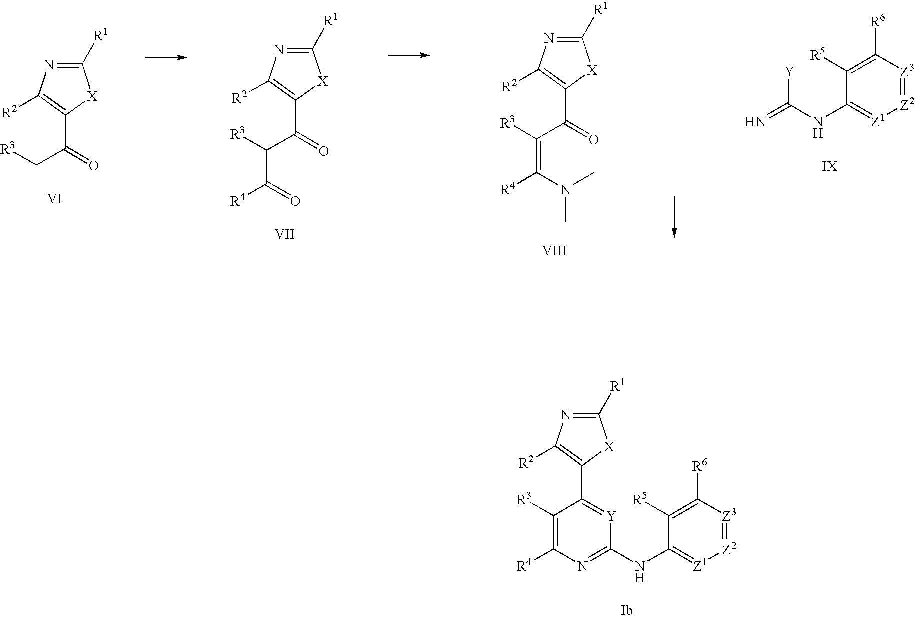
(i) reacting an acylheterocyclic compound of formula VI with R⁴COCl to form a diketone of formula VII;
(ii) converting said diketone of formula VII to a compound of formula VIII;
(iii) reacting said compound of formula VIII with an arylguanidine of formula IX to form said compound of formula Ib.

45. A method of treating a GSK3-dependent disorder, said method comprising administering to a subject in need thereof, a compound of formula Ia, or a pharmaceutically acceptable salt thereof, in an amount sufficient to inhibit GSK3 wherein said compound of formula 1a is: 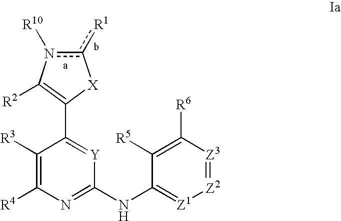
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

46. A method of treating a PLK-dependent disorder, said method comprising administering to a subject in need thereof, a compound of formula Ia, or a pharmaceutically acceptable salt thereof, in an amount sufficient to inhibit PLK, wherein said compound of formula 1a is: 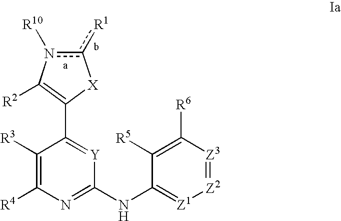
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

47. A method of treating an aurora kinase-dependent disorder, said method comprising administering to a subject in need thereof, a compound of formula Ia, or a pharmaceutically acceptable salt thereof, in an amount sufficient to inhibit aurora kinase wherein said compound of formula 1a is: 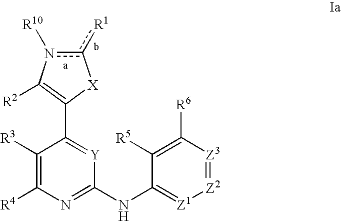
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R¹ is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.

48. A method of treating a CDK-dependent disorder, said method comprising administering to a subject in need thereof, a compound of formula Ia, or a pharmaceutically acceptable salt thereof, in an amount sufficient to inhibit a cyclin dependent kinase, wherein said compound of formula 1a is: 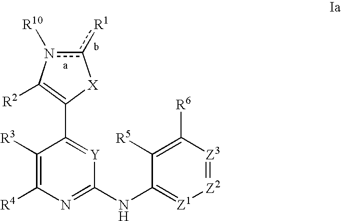
wherein:
(A) “a” is a single bond and “b” is a double bond;
R¹ and R² are each independently as defined below;
R¹⁰ is absent; or
(B) “a” is a double bond and “b” is a single bond;
R¹ is oxygen;
R² is as defined below; and
R¹⁰ is H or alkyl;
X is S, O, NH, or NR⁷;
Y is N or CR⁸;
one of Z¹, Z², and Z³ is N or N⁺R^{a} and the remainder are each independently CR⁷;
R¹, R², R⁵ and R⁶ are each independently R⁷;
R³ and R⁴ are each independently R⁸;
each R⁷ is independently H, halogen, NR^{b}R^{c}, OR^{d} or a hydrocarbyl group optionally substituted by one or more R⁹ groups;
each R⁸ is independently H or (CH₂)ₙR⁹, where n is 0 or 1;
each R⁹ is independently selected from H, halogen, NO₂, CN, R^{e}, NHCOR^{f}, CF₃, COR^{g}, NR^{h}Rⁱ, CONR^{j}R^{k}, SO₂NR^{l}R^{m}, SO₂Rⁿ, OR^{p}, OCH₂CH₂OR^{q}, morpholino, piperidinyl and piperazinyl; and
R^{a-q} are each independently H or alkyl, wherein said alkyl group is optionally substituted by one or more R⁹ groups.
